(19) United States
(12) Patent Application Publication
(10) Pub. No.: US 2017/0281815 A1
(43) Pub. Date: Oct. 5, 2017

(54) Fabric Sterilization Tote Apparatus and Related Methods

(71) Applicant: Symmetry Medical Manufacturing, Inc., Warsaw, IN (US)

(72) Inventor: Steve Hinora, Warsaw, IN (US)

(21) Appl. No.: 15/089,798

(22) Filed: Apr. 4, 2016

(51) Int. Cl. A61L 2/26; A61B 50/31; A61L 2/04

(52) U.S. Cl. CPC A61L 2/26; A61L 2/04; A61B 50/31; A61B 2050/0088

(57) ABSTRACT

A fabric sterilization tote apparatus and related methods of using and manufacturing the same is provided. The apparatus includes a flexible, bacteria-impenetrable material formed into a closable container, the closable container having a base, at least four sidewalls, and a cover, wherein an interior area of the closable container is defined by the base, the at least four sidewalls, and the cover. A bacteria-impenetrable closure is positioned between the cover and at least one of the four sidewalls, wherein the bacteria-impenetrable closure controls a sealing of the interior area of the closable container from an exterior atmosphere. In use, a medical instrument sterilization tray with the at least one medical instrument may be placed within the fabric sterilization tote apparatus after completion of a sterilization process.

### FIELD OF THE DISCLOSURE

The present disclosure is generally related to medical instrument sterilization devices and more particularly is related to a fabric sterilization tote apparatus and related methods.

### BACKGROUND OF THE DISCLOSURE

Proper sterilization and storage of medical instruments is one of the foundations of modern medical care. Without properly sterilized medical instruments, medical procedures are highly susceptible to spreading disease and causing infections. Accordingly, it is crucial for medical instruments to be sterilized successfully after each time they are used and stored in such a way to preserve their sterilized state until they are used in a subsequent medical procedure.

There are varying techniques in the medical industry on how to properly handle medical instruments during and after a sterilization process. For sterilization, one widely used technique is to use an autoclave to subject the instruments to a high-pressure and high-temperature environment, which acts to eliminate all unsterile contaminants. Other sterilization techniques may use chemicals such as ethylene oxide or hydrogen peroxide, steam and gas environments, dry heat environments, or microwave environments, among other techniques.

When an autoclave is used, the medical instruments are retained in sterilization trays throughout the duration of the sterilization process. FIGS. 1A-1B are various illustrations of medical sterilization trays 10, in accordance with the prior art. As shown, sterilization trays 10 are commonly open-topped containers having various grommets or other holding structures 12 positioned on a floor of the tray which are used to hold the medical instrument 14 during the sterilization process. The grommets or holding structures 12 are often designed to precisely fit around differently-sized instruments to allow them to be held stationarily with as much exposed surface area as possible. The sterilization tray 10 may further have apertures 16 or similar structures formed in the floor and/or sidewalls of the tray which allow fluids to drain from the medical instruments 14 during or after the sterilization process. Some sterilization trays 10 may be designed for holding instrument sets or predetermined combinations of medical instruments that support completing a specific surgical procedure, while other sterilization trays 10 may include sterilization baskets. Some sterilization trays 10 may use covers to ensure the medical instruments are not disturbed from their positions in the trays.

While the sterilization trays 10 are used successfully during the sterilization process, they are unable to keep the medical instruments sterilized until their next use, namely due to the apertures within the trays which create numerous open paths from the interior of the tray to the outside atmosphere. As such, the sterilization trays must be housed in some other structure to maintain the sterilized state of the medical instruments therein until they are used. There are a variety of devices in the industry that can achieve this task, but they generally fall into two main categories: (1) medical sterilization cases which house the sterilization trays during and after the autoclave process; and (2) sterile wraps which are applied to the sterilization trays immediately after they exit the autoclave.

FIGS. 2A-2E are various illustrations of medical sterilization cases 20, in accordance with the prior art. As shown in FIGS. 2A-2E, the medical sterilization case 20 is generally characterized as a container that is designed to hold the entire sterilization tray 10 (or multiple sterilization trays 10) within an interior space that can be kept sterile. Medical sterilization cases 20 are often solid-walled structures which have a removable cover 22 that can be sealed against a base 24 to prevent bacteria, air, or other substances from gaining access to the interior of the sterilization case 20. FIGS. 2A-2B illustrate side views of the medical sterilization case 20 with a cover 22 positioned on the case body 24. FIG. 2C, for example, illustrates an open medical sterilization case 20, with the cover 22 removed from the body 24, and with the sterilization tray 10 partially positioned in the sterilization case 20. Many sterilization cases have one or more heat-sensitive valves 26 positioned in the cover 22 which opens when the sterilization case 20 is in the high-temperature environment of the autoclave but closes as the autoclave cools down, such that when the sterilization process is complete, the interior of the sterilization case 20 is fully sealed. FIGS. 1D-1E illustrate in detail the heat-sensitive valves 26 which are positioned in the cover 22 of the sterilization case 20.

FIGS. 3A-3D are various illustrations of medical sterilization trays 10 being wrapped in medical sterilization wraps 30, in accordance with the prior art. Sterilization wraps 30 are generally disposable fabrics having a bacteria-resistant construction which are applied to the sterilization trays 10 before they enter the autoclave and remain on the sterilization trays 10 throughout the duration of the sterilization process. In contrast to sterilization cases (FIGS. 2A-2E) which can simply be closed around a sterilization tray 10 to keep it in a sterilized environment, sterilization wraps 30 must be folded around the sterilization trays 10 in a specific manner so the sterilization tray 10 remains sterile, e.g., ensuring that all seams and folds of the sterilization wrap 30 are tight enough to seal out bacteria. Once the sterilization tray 10 is fully wrapped, the sterilization wrap 30 is usually taped in place. FIG. 3A shows the sterilization wrap 30 in a folded position about some sterilization trays (not visible) and in a partially folded position around other sterilization trays 10. FIG. 3B illustrates the sterilization wrap 30 fully folded and taped. FIG. 3C illustrates a sterilization tray 10 with a sterilization wrap 30 partially folded around it. As can be seen in each of these figures, the sterilization wrap 30 is folded about the sterilization tray 10 in a specific, orderly manner to ensure that the edges, corners, and seams of the sterilization wrap 30 do not allow bacteria to gain access to the sterilization tray 10. The specific folding patterns may include those required by ANSI/AAMI ST79, ANSI/AAMI ST41, AORN Standards, or similar standards. After wrapping is complete, the wrapped sterilization tray 10 sterilized and then stored on a shelf 32, as shown in FIG. 3D, until the medical instruments therein are required in a medical procedure.

Sterilization cases are recognized in the industry as being durable, reliable, and generally easy to use, but they are also well-known to be bulky and expensive. The sterilization cases must be large enough to hold the sterilization tray which means their overall dimensions are significantly larger than the sterilization tray itself, as is visible in FIGS. 2A-2E. The storage rooms and operating rooms of medical facilities have limited storage abilities, so the size constraint of the sterilization cases complicates their use. Further, sterilization cases can cost upwards of $700 per unit, which is significantly higher than sterilization wraps, often costing between $10 and $100 per unit.

Sterilization wraps, on the other hand, are able to be closely folded around sterilization trays, so storage space is less of an issue. And, sterilization wraps are generally far less expensive to initially purchase than sterilization cases. However, sterilization wraps have many shortcomings. For one, they are intended to be disposable which is costly over a long period of time and disposing of them after one use is wasteful. Further, sterilization wraps must be folded properly around the sterilization trays to ensure the instruments remain sterilized. This folding must be carefully taught to sterilization technicians which requires substantial training. Even then, improper folding can still occur due to human error. In addition, sterilization wraps are not made from highly durable materials so they are susceptible to being torn or ripped, especially when being transported or positioned on a shelf. It is not uncommon in the industry for a sterilization wrap to be torn by the shelf it is being placed on. One final aspect of sterilization wraps is that they have a short life span for successfully keeping the sterilization tray sterile. While sterilization cases may be able to be stored for weeks, sterilization wraps may only be capable of being used successfully for a few days.

Thus, a heretofore unaddressed need exists in the industry to address the aforementioned deficiencies and inadequacies.

### SUMMARY OF THE DISCLOSURE

Embodiments of the present disclosure provide a fabric sterilization tote apparatus as well as related systems and methods thereof. Briefly described, in architecture, one embodiment of the apparatus, among others, can be implemented as follows. A flexible, bacteria-impenetrable material is formed into a closable container, the closable container having a base, at least four sidewalls, and a cover, wherein an interior area of the closable container is defined by the base, the at least four sidewalls, and the cover. A bacteria-impenetrable closure is positioned between the cover and at least one of the four sidewalls, wherein the bacteria-impenetrable closure controls a sealing of the interior area of the closable container from an exterior atmosphere.

The present disclosure can also be viewed as providing methods for sterilizing medical instruments and storing sterilized medical instruments thereafter. In this regard, one embodiment of such a method, among others, can be broadly summarized by the following steps: placing a medical instrument sterilization tray with the at least one medical instrument in a fabric sterilization tote apparatus, wherein the fabric sterilization tote apparatus comprises: a flexible, bacteria-impenetrable material formed into a closable container, the closable container having a base, at least four sidewalls, and a cover, wherein an interior area of the closable container is defined by the base, the at least four sidewalls, and the cover; and a bacteria-impenetrable closure positioned between the cover and at least one of the four sidewalls, wherein the bacteria-impenetrable closure controls a sealing of the interior area of the closable container from an exterior atmosphere; sterilizing the fabric sterilization tote apparatus with medical instrument sterilization tray and the at least one medical instrument in an autoclave; and removing the fabric sterilization tote apparatus with medical instrument sterilization tray and the at least one medical instrument from the autoclave, whereby the interior area of the closable container remains sterile.

The present disclosure can also be viewed as providing methods of manufacturing a fabric sterilization tote apparatus. In this regard, one embodiment of such a method, among others, can be broadly summarized by the following steps: forming a closable container from a flexible, bacteria-impenetrable material, wherein the closable container has a base, at least four sidewalls, and a cover, and wherein an interior area of the closable container is defined by the base, the at least four sidewalls, and the cover; and integrating a bacteria-impenetrable closure between the cover and at least one of the four sidewalls, thereby controlling a sanitized environment within the interior area by sealing the interior area of the closable container from an exterior atmosphere with the bacteria-impenetrable closure.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1B are various illustrations of medical sterilization trays, in accordance with the prior art.

FIGS. 2A-2E are various illustrations of medical sterilization cases, in accordance with the prior art.

FIGS. 3A-3D are various illustrations of medical sterilization trays being wrapped in medical sterilization wraps, in accordance with the prior art.

FIG. 4 is an elevated front view illustration of a fabric sterilization tote apparatus, in accordance with a first exemplary embodiment of the present disclosure.

FIG. 5 is an elevated front view illustration of the fabric sterilization tote apparatus of FIG. 4 with a zipper-based bacteria-impenetrable closure having two slides, in accordance with a first exemplary embodiment of the present disclosure.

FIGS. 6A-6C are first side, bottom, and second side view illustrations of the fabric sterilization tote apparatus of FIG. 4 with reinforcing strap, in accordance with a first exemplary embodiment of the present disclosure.

FIG. 7 is an isometric view illustration of the fabric sterilization tote apparatus of FIG. 4 with a corner reinforcing member, in accordance with a first exemplary embodiment of the present disclosure.

FIG. 8 is a cross-sectional view illustration of the fabric sterilization tote apparatus of FIG. 4 with multiple sterilization trays positioned in an interior area, in accordance with a first exemplary embodiment of the present disclosure.

FIG. 9 is a flowchart illustrating a method for sterilizing medical instruments and storing sterilized medical instruments thereafter, in accordance with the first exemplary embodiment of the present disclosure.

FIG. 10 is a flowchart illustrating a method of manufacturing a fabric sterilization tote apparatus, in accordance with the first exemplary embodiment of the present disclosure.

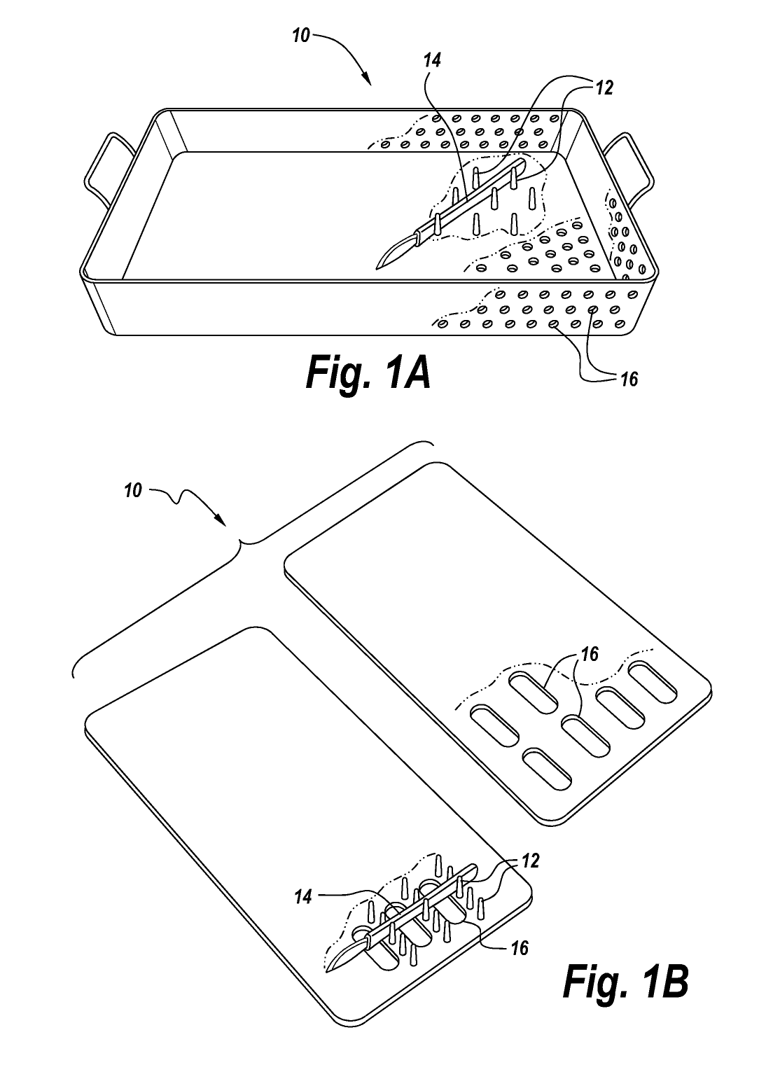

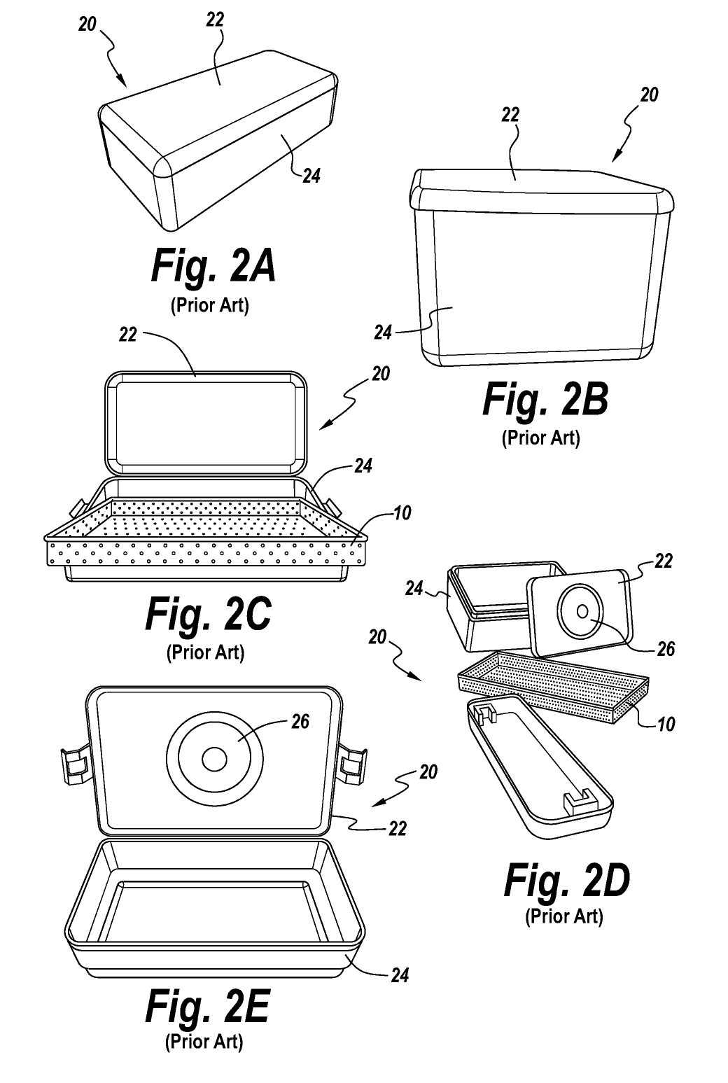

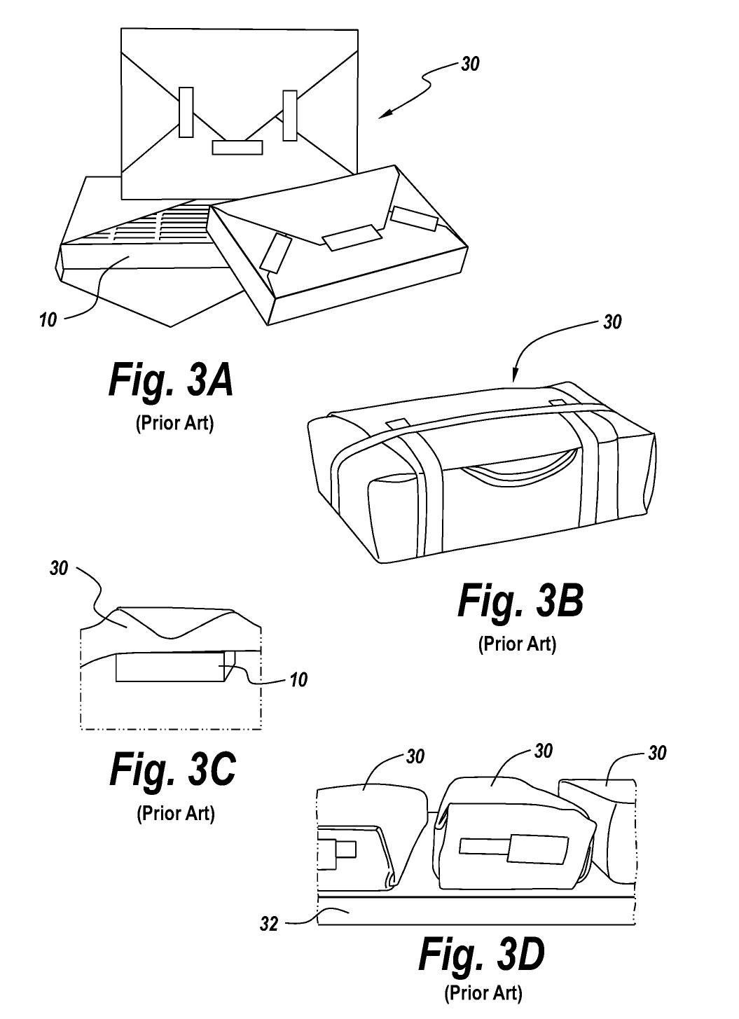

### DETAILED DESCRIPTION

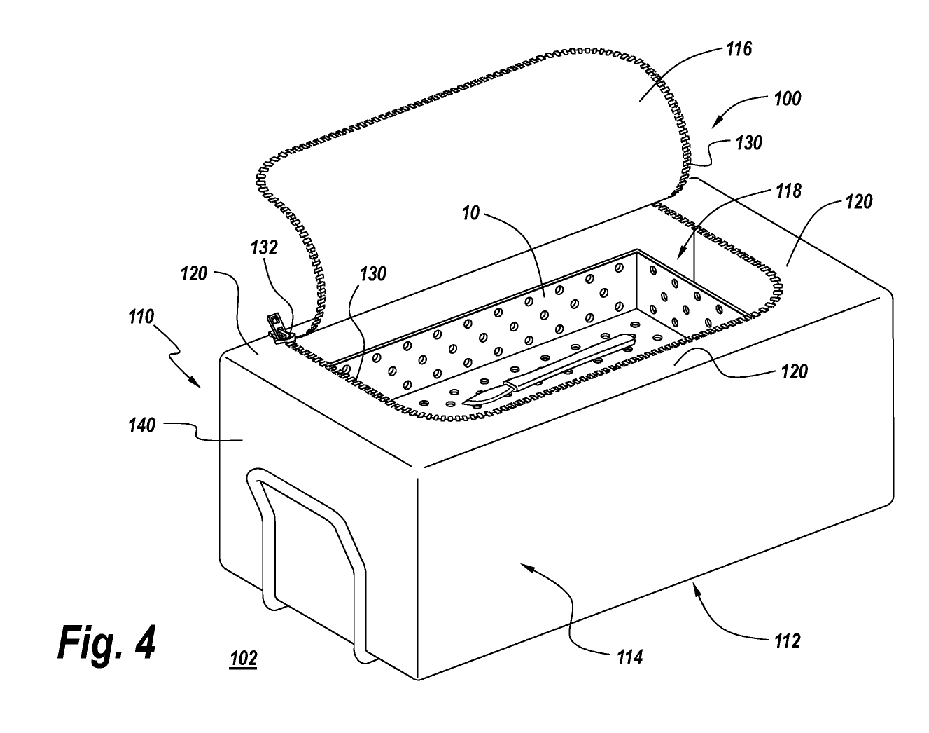

FIG. 4 is an elevated front view illustration of a fabric sterilization tote apparatus 100, in accordance with a first exemplary embodiment of the present disclosure. The fabric sterilization tote apparatus 100, which may be referred to herein as ‘apparatus 100’ includes a flexible, bacteria-impenetrable material formed into a closable container 110, the closable container 110 having a base 112, at least four sidewalls 114, and a cover 116. An interior area 118 of the closable container 110 is defined by the base 112, the at least four sidewalls 114, and the cover 116. A bacteria-impenetrable closure 130 is positioned between the cover 116 and at least one of the four sidewalls 114. The bacteria-impenetrable closure 130 controls a sealing of the interior area 118 of the closable container 110 from an exterior atmosphere 102.

The apparatus 100 may be used during a medical instrument sterilization process and thereafter to ensure that medical instruments are properly sterilized and remain sterile until they are used in a medical procedure. Relative to the Background of this disclosure, the apparatus 100 may be used to fully replace conventional medical sterilization cases and fully replace conventional medical sterilization wraps. The apparatus 100 may prove to be far more efficient than manually wrapping a medical instrument tray with a sterilization wrap and it may provide a more cost-effective solution than the conventional metal sterilization case. Accordingly, the apparatus 100 may overcome the shortcomings of both conventional devices and provide additional benefits within the field of medical sterilization.

The apparatus 100 may be formed from a flexible, bacteria-impenetrable material, such as a sterilizing paper, a non-woven fabric such as spun-melt-spun (SMS) polypropylene, or other flexible fabric or paper-like substrates that are bacteria-impenetrable. Unlike conventional sterilization wraps which are constructed as flat sheets that are folded around a sterilization tray, the flexible, bacteria-impenetrable material of the apparatus 100 may be formed into a closable container 110 capable of housing the sterilization tray 10 carrying medical instruments (as shown in FIG. 4) or medical instruments without a sterilization tray. In general terms, the closable container 110 may be viewed as a tote, in that, it is a structured container that is capable of removably housing sterilization trays 10 in an enclosable interior area 118.

The closable container 110 may generally have a base 112, at least four sidewalls 114, and a cover 116, although different configurations are envisioned as well. The cover 116 may generally be positioned at a top side of the closable container 110, but covers 116 positioned on a vertical sidewall are also possible. The shape and dimensions of the closable container 110 may be selected to substantially match the sterilization tray 10 or trays which the apparatus 100 is intended to hold, leaving enough tolerance between the base 112, sidewalls 114, and cover 116 and the sterilization tray 10. The interior area 118 of the closable container 110 may be defined by the space that the base 112, the at least four sidewalls 114, and the cover 116 shape when the cover 116 has a closed position to form an area fully interior of the base 112, sidewalls 114, and cover 116. The cover 116 may form the full portion of a side of the closable container 110 or it may form a portion of a side of the closable container 110, as shown in FIG. 4, where the top side of the closable container 110 has edges 120 which are engagable with the cover 116.

The bacteria-impenetrable closure 130 is positioned between the cover 116 and at least one of the four sidewalls 114 to allow the cover 116 to be secured in a closed position. As shown in FIG. 4, the bacteria-impenetrable closure 130 may be positioned about a number of sides of the cover 116, such as along extended edges of the sidewalls 114 which form the edges 120 of the top-facing side of the closable container 110. The bacteria-impenetrable closure 130 controls a sealing of the interior area 118 of the closable container 110 from an exterior atmosphere 102, thereby allowing the interior area 118 to be fully separated from the exterior atmosphere 102.

The bacteria-impenetrable closure 130 may be a bacteria-proof zipper which has teeth positioned along the edges of the cover 116 and the interior terminating ends of the edges 120 on the topside of the closable container 110. The zipper may include any number of sliders 132 which are capable of engaging and disengaging the teeth of the zipper. In one example, two sliders may be used, thereby allowing for the bacteria-impenetrable closure 130 to be closed from multiple directions.

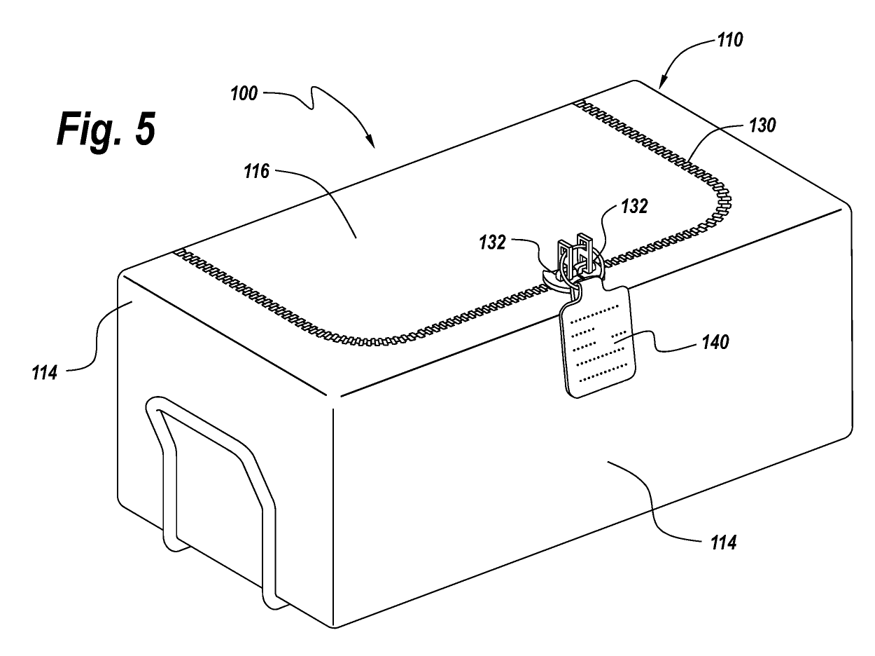

FIG. 5 is an elevated front view illustration of the fabric sterilization tote apparatus 100 of FIG. 4 with a zipper-based bacteria-impenetrable closure 130 having two slides 132, in accordance with a first exemplary embodiment of the present disclosure. As shown, the two slides 132 may be positioned in an abutting position to close the bacteria-impenetrable closure 130 fully. When this configuration of the two slides 132 is achieved, a tag 140 may be connected between the two slides 132. The tag 140 may be an identification tag which has various identifying information about the apparatus 100 or the contents thereof, such as type of medical instruments, date of initial sterilization, expiration date of sterilization, etc. Positioning the tag 140 between the two slides 132 of the zipper may ensure that the zipper remains closed, thereby ensuring that the interior area of the closable container 110 remains sterile.

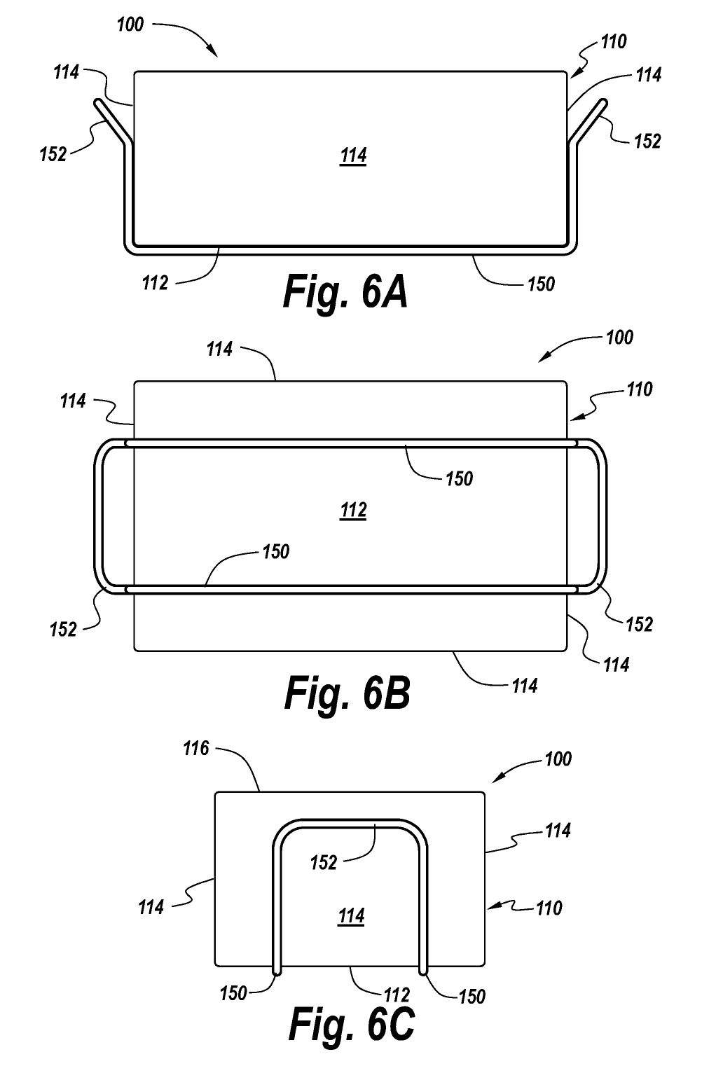

FIGS. 6A-6C are first side, bottom, and second side view illustrations of the fabric sterilization tote apparatus 100 of FIG. 4 with reinforcing strap 150, in accordance with a first exemplary embodiment of the present disclosure. FIGS. 6A-6C illustrate a reinforcing strap 150 which may be used to reinforce the flexible, bacteria-impenetrable material from which the closable container 110 is formed from, thereby allowing the apparatus 100 to be carried or otherwise transported without tearing the flexible, bacteria-impenetrable material. The reinforcing strap 150 may include a durable material which can be integrated with the flexible, bacteria-impenetrable material, such as a nylon string material which can be affixed to the flexible, bacteria-impenetrable material with an adhesive or similar bonding means. The reinforcing strap 150 may be positioned across at least a portion of the base 112 and at least two of the at least four sidewalls 114, thereby allowing the reinforcing strap 150 to receive the weight of the apparatus 100 when it is being used and allowing a user to carry the apparatus 100 from its sides. In one example, the reinforcing strap 150 may be a single length of material which is bonded to the flexible, bacteria-impenetrable material along the base 112 and a portion of the sidewalls 114 yet has a length that is not bonded to the flexible, bacteria-impenetrable material and extends away from the sidewalls 114 to form a handle 152.

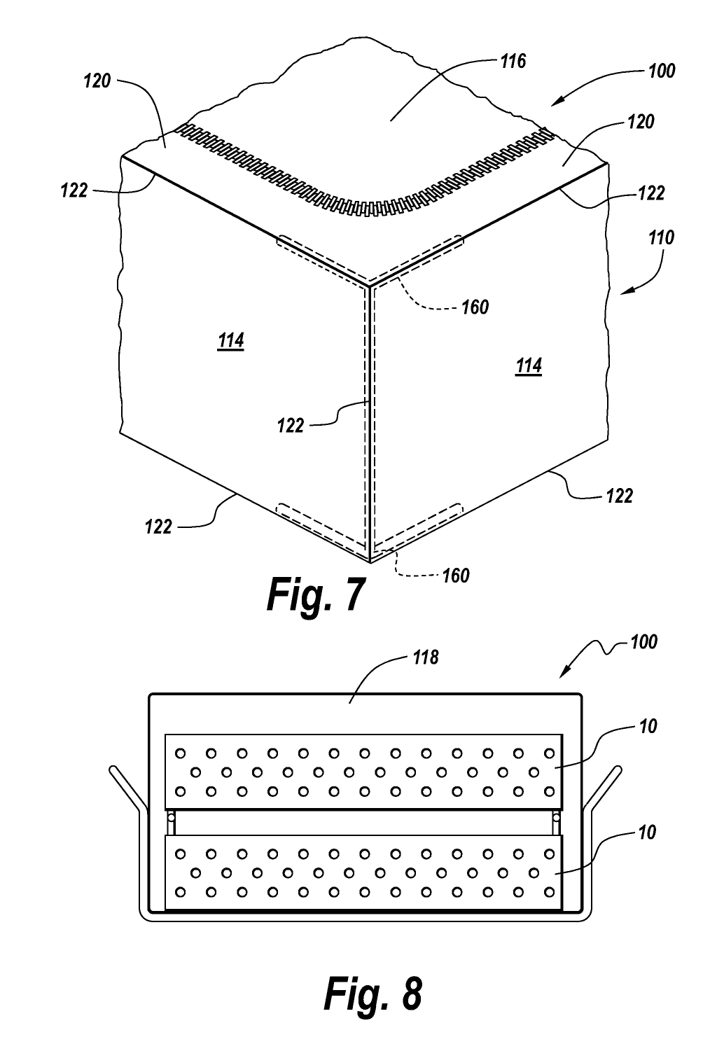

FIG. 7 is an isometric view illustration of the fabric sterilization tote apparatus 100 of FIG. 4 with a corner reinforcing member 160, in accordance with a first exemplary embodiment of the present disclosure. The reinforcing member 160 may be a structure which is integrated into the apparatus 100 to help the closable container 110 retain its general shape. For example, the reinforcing member 160 may be used to keep the interior area of the closable container 110 substantially constant throughout its use. In one example, the reinforcing member 160 includes plastic tubing or similar materials which are positioned at a corner 122 between two of the at least four sidewalls 114. The reinforcing member 160 may also be positioned at a corner between at least one of the four sidewalls 114 and at least one of the base 112 and cover 116. The reinforcing member 160 may be positioned on an interior or exterior of the closable container 110 with any type of fastener or fastening system. It may be preferable for the reinforcing member 160 to be positioned along an entirety of the corner 122 between adjacent sidewalls 114 yet on only a portion of the corner 122 between sidewalls 114 and the cover 116 and/or base 112.

FIG. 8 is a cross-sectional view illustration of the fabric sterilization tote apparatus 100 of FIG. 4 with multiple sterilization trays 10 positioned in an interior area 118, in accordance with a first exemplary embodiment of the present disclosure. As shown, the apparatus 100 may be sized large enough to carry two or more sterilization trays 10 within the interior area 118. The number of sterilization trays 10 that the apparatus 100 is capable of holding may be dependent on the weight of the sterilization trays 10, the weight of the medical instruments positioned in the sterilization trays 10, and/or the relationship between the medical instruments. For example, it may be common for the apparatus 100 to hold two sterilization trays 10 which are both required for a single medical procedure.

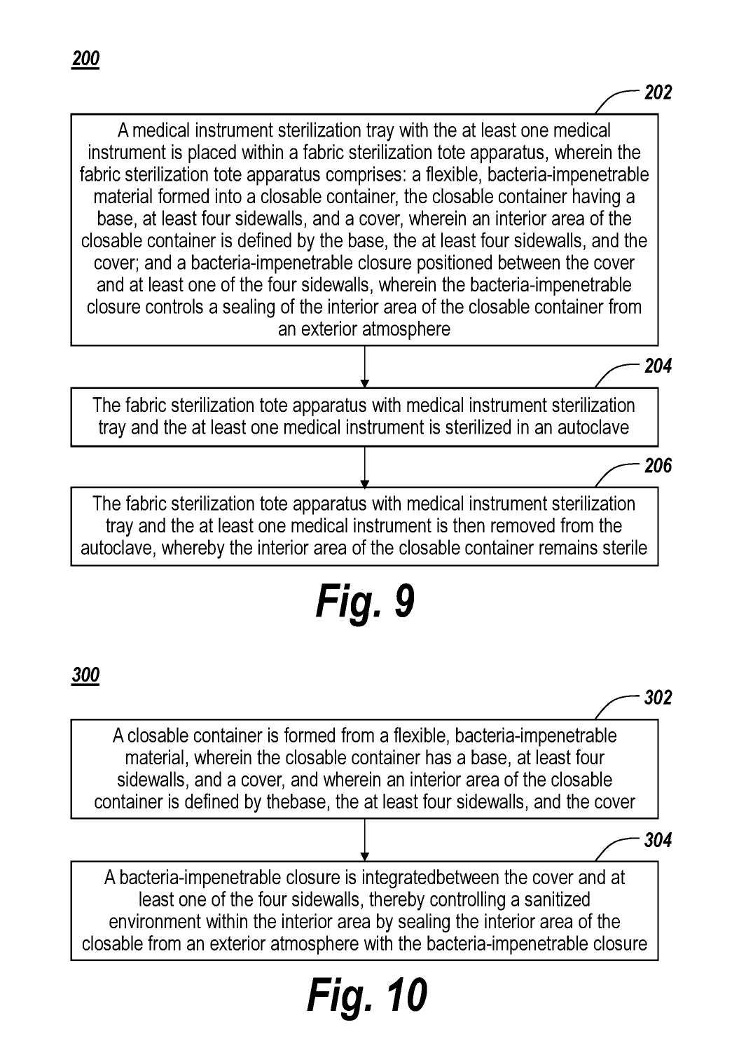

FIG. 9 is a flowchart 200 illustrating a method for sterilizing medical instruments and storing sterilized medical instruments thereafter, in accordance with the first exemplary embodiment of the present disclosure. It should be noted that any process descriptions or blocks in flow charts should be understood as representing modules, segments, or steps that include one or more instructions for implementing specific logical functions in the process, and alternate implementations are included within the scope of the present disclosure in which functions may be executed out of order from that shown or discussed, including substantially concurrently or in reverse order, depending on the functionality involved, as would be understood by those reasonably skilled in the art of the present disclosure.

As is shown by block 202, a medical instrument sterilization tray with the at least one medical instrument is placed within a fabric sterilization tote apparatus, wherein the fabric sterilization tote apparatus comprises: a flexible, bacteria-impenetrable material formed into a closable container, the closable container having a base, at least four sidewalls, and a cover, wherein an interior area of the closable container is defined by the base, the at least four sidewalls, and the cover; and a bacteria-impenetrable closure positioned between the cover and at least one of the four sidewalls, wherein the bacteria-impenetrable closure controls a sealing of the interior area of the closable container from an exterior atmosphere. The fabric sterilization tote apparatus with medical instrument sterilization tray and the at least one medical instrument is sterilized in an autoclave (block 204). The fabric sterilization tote apparatus with medical instrument sterilization tray and the at least one medical instrument is then removed from the autoclave, whereby the interior area of the closable container remains sterile (block 206). The interior area may remain sterile for a period of time long enough to allow the medical instruments to be used in a future medical procedure.

It is noted that the method may include any number of additional steps, processes, or functions, including any disclosed relative to any other figure of this disclosure. For example, prior to insertion into the autoclave, the cover of the closable container may be closed with the bacteria-impenetrable closure. In one example, this closure may consist of simply moving at least one slider of a zipper along teeth of the zipper, which is far more efficient and secure than the conventional folding process with conventional sterilization wraps. Accordingly, the cover of the closable container can be fully closed with the bacteria-impenetrable closure without any need to fold or tape the flexible, bacteria-impenetrable material. Since no folding of the flexible, bacteria-impenetrable material is required, the corners of the apparatus may be formed from a single layer of the flexible, bacteria-impenetrable material. The method may also include transporting the sterilized fabric sterilization tote apparatus with at least two handles formed by at least one reinforcing strap positioned across at least a portion of the base and at least two of the at least four sidewalls.

FIG. 10 is a flowchart 300 illustrating a method of manufacturing a fabric sterilization tote apparatus, in accordance with the first exemplary embodiment of the present disclosure. It should be noted that any process descriptions or blocks in flow charts should be understood as representing modules, segments, portions of code, or steps that include one or more instructions for implementing specific logical functions in the process, and alternate implementations are included within the scope of the present disclosure in which functions may be executed out of order from that shown or discussed, including substantially concurrently or in reverse order, depending on the functionality involved, as would be understood by those reasonably skilled in the art of the present disclosure.

As is shown by block 302, a closable container is formed from a flexible, bacteria-impenetrable material, wherein the closable container has a base, at least four sidewalls, and a cover, and wherein an interior area of the closable container is defined by the base, the at least four sidewalls, and the cover. A bacteria-impenetrable closure is integrated between the cover and at least one of the four sidewalls, thereby controlling a sanitized environment within the interior area by sealing the interior area of the closable container from an exterior atmosphere with the bacteria-impenetrable closure (block 304). It is noted that the method may include any number of additional steps, processes, or functions, including any disclosed relative to any other figure of this disclosure. For example, the closable container may be reinforced with a reinforcing strap positioned across at least a portion of the base and at least two of the at least four sidewalls.

As noted previously, the apparatus 100 may offer a number of benefits over the conventional art within the medical sterilization industry, such as being more efficient than manually wrapping a medical instrument tray with a sterilization wrap and being more cost-effective than the conventional metal sterilization case. It is further noted that the apparatus 100 may be easier and more efficient to store in an instrument storage room or operating room than conventional sterilization cases and it may offer a longer sterilized shelf life than conventional sterilization wraps.

Another benefit of the apparatus 100 is it may reduce the risk of wet pack problems in sterilization. Wet packs is a phenomenon in the medical sterilization industry where moisture remains in an autoclave or the sterilized items themselves after the autoclave process. Wet packs can cause contamination issues because excessive moisture can act as a pathway for microorganisms. This can lead to re-contaminating the sterilized instruments. In others words, if the sterilized items are not properly dried then microorganisms may wick through the wrapping and deposit themselves on the sterile load. While wet packs can occur for a number of reasons, one reason is believed to be cooling issues with metal sterilization enclosures, namely between conventional metal sterilization cases and the metal sterilization trays which reside inside the sterilization cases. By eliminating the need for the conventional sterilization case through the use of the apparatus 100, it may be possible to lessen the prevalence of wet packs.

It should be emphasized that the above-described embodiments of the present disclosure, particularly, any “preferred” embodiments, are merely possible examples of implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and the present disclosure and protected by the following claims.

What is claimed is:

1. A fabric sterilization tote apparatus comprising:
a flexible, bacteria-impenetrable material formed into a closable container, the closable container having a base, at least four sidewalls, and a cover, wherein an interior area of the closable container is defined by the base, the at least four sidewalls, and the cover; and
a bacteria-impenetrable closure positioned between the cover and at least one of the four sidewalls, wherein the bacteria-impenetrable closure controls a sealing of the interior area of the closable container from an exterior atmosphere.

2. The fabric sterilization tote apparatus of claim 1, wherein the bacteria-impenetrable closure further comprises a zipper having at least one slider.

3. The fabric sterilization tote apparatus of claim 2, wherein the zipper further comprises two sliders, and wherein an identification tag is removably connected between the two sliders.

4. The fabric sterilization tote apparatus of claim 1, further comprising a reinforcing member positioned at a corner between two of the at least four sidewalls.

5. The fabric sterilization tote apparatus of claim 4, wherein the reinforcing member further comprises substantially rigid plastic tubing.

6. The fabric sterilization tote apparatus of claim 1, further comprising a reinforcing member positioned at a corner between at least one of the four sidewalls and at least one of the base and cover.

7. The fabric sterilization tote apparatus of claim 1, further comprising a reinforcing strap positioned across at least a portion of the base and at least two of the at least four sidewalls.

8. The fabric sterilization tote apparatus of claim 7, wherein the reinforcing strap further comprises a nylon material.

9. The fabric sterilization tote apparatus of claim 7, wherein the reinforcing strap further comprises handles positioned along at least two of the at least four sidewalls.

10. The fabric sterilization tote apparatus of claim 1, wherein the flexible, bacteria-impenetrable material further comprises at least one of a sterilization paper and a sterilization non-woven fabric.

11. A method for sterilizing medical instruments and storing sterilized medical instruments thereafter, the method comprising:
placing a medical instrument sterilization tray with the at least one medical instrument in a fabric sterilization tote apparatus, wherein the fabric sterilization tote apparatus comprises:
a flexible, bacteria-impenetrable material formed into a closable container, the closable container having a base, at least four sidewalls, and a cover, wherein an interior area of the closable container is defined by the base, the at least four sidewalls, and the cover; and
a bacteria-impenetrable closure positioned between the cover and at least one of the four sidewalls, wherein the bacteria-impenetrable closure controls a sealing of the interior area of the closable container from an exterior atmosphere;
sterilizing the fabric sterilization tote apparatus with medical instrument sterilization tray and the at least one medical instrument in an autoclave; and
removing the fabric sterilization tote apparatus with medical instrument sterilization tray and the at least one medical instrument from the autoclave, whereby the interior area of the closable container remains sterile.

12. The method of claim 11, further comprising closing the cover of the closable container with the bacteria-impenetrable closure.

13. The method of claim 12, wherein the bacteria-impenetrable closure further comprises a zipper having at least one slider.

14. The method of claim 13, wherein closing the cover of the closable container with the bacteria-impenetrable closure consists of moving the at least one slider along teeth of the zipper.

15. The method of claim 12, wherein closing the cover of the closable container with the bacteria-impenetrable closure is free from folding and taping the flexible, bacteria-impenetrable material.

16. The method of claim 12, further comprising transporting the sterilized fabric sterilization tote apparatus with at least two handles formed by at least one reinforcing strap positioned across at least a portion of the base and at least two of the at least four sidewalls.

17. The method of claim 11, wherein the closable container further comprises a reinforcing member positioned at a corner between two of the at least four sidewalls.

18. The method of claim 17, wherein the corner between two of the at least four sidewalls is formed from a single layer of the flexible, bacteria-impenetrable material.

19. A method of manufacturing a fabric sterilization tote apparatus, the method comprising:
forming a closable container from a flexible, bacteria-impenetrable material, wherein the closable container has a base, at least four sidewalls, and a cover, and wherein an interior area of the closable container is defined by the base, the at least four sidewalls, and the cover; and
integrating a bacteria-impenetrable closure between the cover and at least one of the four sidewalls, thereby controlling a sanitized environment within the interior area by sealing the interior area of the closable container from an exterior atmosphere with the bacteria-impenetrable closure.

20. The method of claim 19, further comprising the step of reinforcing the closable container with a reinforcing strap positioned across at least a portion of the base and at least two of the at least four sidewalls.
